(19) United States
(12) Patent Application Publication
(10) Pub. No.: US 2023/0233312 A1
(43) Pub. Date: Jul. 27, 2023

(54) STENT DESIGN FOR TRANSLUMINAL APPLICATION

(71) Applicant: Covidien LP, Mansfield, MA (US)

(72) Inventors: Siddhi K. Desai, Sunnyvale, CA (US); Ailee T. Pham, Sunnyvale, CA (US)

(21) Appl. No.: 17/581,654

(22) Filed: Jan. 21, 2022

(51) Int. Cl. A61F 2/04; A61M 27/00

(52) U.S. Cl. CPC A61F 2/04; A61M 27/008; A61F 2002/041; A61F 2220/0008

(57) ABSTRACT

Apparatuses, and systems are described for stent designs for transluminal application. The stent may include a stent body having a diameter and a length in a deployed configuration. The stent may include a helical wrapping pattern that is at least partially covered with a material. The helical wrapping pattern may be configured to reduce a foreshortening of the stent body upon deployment from an undeployed configuration to the deployed configuration to less than ten percent of a length of the stent body in the undeployed configuration. In some cases, the stent may include a first anchoring member coupled with a distal portion of the stent body and a second anchoring member coupled with a proximal portion of the stent body. The first and second anchoring members may be configured to increase a diameter of the stent.

### BACKGROUND

Diseases and disorders of the gallbladder, pancreas, and bile ducts (i.e., pancreaticobiliary system) are associated with significant morbidity, mortality, and impaired quality of life. Obstructions, tumors, injuries, leakages, inflammation, infection, and lesions can occur in these structures, which can eventually lead to conditions such as biliary colic, cholecystitis, choledocholithiasis, cholelithiasis, pancreatitis, pancreatic duct stone formations, and chronic abdominal pain. Diseases of the pancreaticobiliary system may also be associated with nutritional disorders, such as malnutrition, obesity, and high cholesterol.

To treat a biliary obstruction, a clinician may perform a stent delivery procedure to place a stent across the body lumen to bypass the obstruction. In general, a stent delivery procedure may include placing an endoscope into the gastrointestinal tract and accessing the bile duct with a catheter. A guidewire may then be deployed through the catheter and into the bile duct. Once the guidewire is in place, a stent or other treatment device may be advanced over the guidewire into the bile duct. After the stent is placed in the bile duct, the clinician may withdraw the stent delivery system.

### SUMMARY

The stent designs for transluminal application may include a stent body having a first diameter and a first length in a deployed configuration and including a helical wrapping pattern that is at least partially covered with a first material. In some case, the helical wrapping pattern may be configured to reduce a foreshortening of the stent body upon deployment from an undeployed configuration to the deployed configuration to less than ten percent of a length of the stent body in the undeployed configuration. The stent may further include a first anchoring member coupled with a distal portion of the stent body and configured to increase a diameter of the distal portion of the stent body to a second diameter greater than the first diameter. In some examples, the stent may include a second anchoring member coupled with a proximal portion of the stent body and configured to increase a diameter of the proximal portion of the stent body to the second diameter greater than the first diameter.

A stent may be described. The stent may include a stent body having a first diameter in a deployed configuration, a first length in the deployed configuration, and comprising a helical wrapping pattern that is at least partially covered with a first material, wherein the helical wrapping pattern is configured to reduce a foreshortening of the stent body upon deployment from an undeployed configuration to the deployed configuration to less than ten percent of a length of the stent body in the undeployed configuration, a first anchoring member coupled with a distal portion of the stent body and configured to increase a diameter of the distal portion of the stent body to a second diameter greater than the first diameter, and a second anchoring member coupled with a proximal portion of the stent body and configured to increase a diameter of the proximal portion of the stent body to the second diameter greater than the first diameter.

In some cases, the first anchoring member comprises a first flared portion coupled with a distal end of the stent body and spaced around a circumference of the distal end of the stent body, and wherein the second anchoring member comprises a second flared portion coupled with a proximal end of the stent body and spaced around a circumference of the proximal end of the stent body. In some cases, a length the first anchoring member is different than a length of the second anchoring member. In some cases, a length the first anchoring member is the same as a length of the second anchoring member.

In some cases, the first material is disposed onto an entire portion of the stent body, the first anchoring member, and the second anchoring member. In some cases, the helical wrapping pattern comprises a single wire frame. In some cases, the first material comprises a plurality of drainage holes disposed within the second anchoring member and the proximal portion of the stent body. The stent may further include one or more markers disposed around the stent body, the first anchoring member, and the second anchoring member. In some cases, the helical wrapping pattern comprises more than one wire frame. In some cases, the helical wrapping pattern comprises a laser cut frame.

In some cases, the stent body further comprises a braided wrapping pattern coupled with a distal end of the helical wrapping pattern, wherein the braided wrapping pattern comprises a braided frame. In some cases, the helical wrapping pattern comprises a single wire frame. In some cases, the first anchoring member comprises a flared portion coupled with the distal end of the stent body and spaced around a circumference of the distal end of the stent body, wherein the first anchoring member comprises the braided wrapping pattern. In some cases, the braided wrapping pattern is uncovered from the first material.

In some cases, the helical wrapping pattern comprises a first wire frame around a first portion of a circumference of the stent body and a second wire frame around a second portion of the circumference of the stent body, wherein the first wire frame and the second wire frame are connected across the length of the stent body. In some cases, the first anchoring member comprises a fast fin that protrudes from the distal portion of the stent body and extends in a proximal direction or a distal direction, and wherein the second anchoring member comprises a second fin that protrudes from the proximal portion of the stent body and extends in the proximal direction or the distal direction. In some cases, the first material is disposed onto an entire portion of the stent body, the first anchoring member, and the second anchoring member.

Certain embodiments of the present disclosure may include some, all, or none of the above advantages or features. One or more other technical advantages or features may be readily apparent to those skilled in the art from the figures, descriptions, and claims included herein. Moreover, while specific advantages or features have been enumerated above, various embodiments may include all, some, or none of the enumerated advantages or features.

Further scope of the applicability of the described methods and systems will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates a system for providing access to a body lumen in accordance with aspects of the present disclosure.

FIG. 2 illustrates a stent with flared portions in accordance with aspects of the present disclosure.

FIG. 3 illustrates a stent with more than one wire frame in accordance with aspects of the present disclosure.

FIG. 4 illustrates a stent with anchoring fins in accordance with aspects of the present disclosure.

FIG. 5 illustrates a hybrid stent in accordance with aspects of the present disclosure.

FIG. 6 illustrates a stent delivery system with the stent fully deployed in accordance with aspects of the present disclosure.

### DETAILED DESCRIPTION

The present disclosure is generally directed to stent designs for transluminal application. In certain procedures described herein, to place a stent within a body lumen, the luminal wall is pierced, and a stent delivery system is advanced through the hole (i.e., access site or access hole) and positioned at the target site to bypass an obstruction. The stent is then deployed from the stent delivery system. If the stent is not accurately and precisely deployed, the stent may be unable to form a bridge between two body lumens and therefore, may be unable to bypass the obstruction. For example, if the distal portion of the stent is deployed short of (e.g., below) the access hole, the stent may be unable to connect the two body lumens and unable to form an alternate route to bypass the obstruction. In some cases, inaccurate deployment may result in fluid from the lumen leaking out into the surrounding tissue and organs, which may potentially cause serious discomfort or other medical complications.

To address these concerns and other deficiencies, the stent placed within the body lumen may be an example of a non-foreshortening stent. For example, the stent may include a helical wrapping pattern that may be configured to reduce a foreshortening of the stent body upon deployment from the undeployed configuration to the deployed configuration to less than ten percent of a length of the stent body in the undeployed configuration. In such cases, the stent may remain in place during retraction of the outer sheath and deployment of the distal portion as the outer sheath continues to be retracted proximally. The non-foreshortening stent may enable accurate deployment by positioning the stent within the body lumen and maintaining the position of the stent within the body lumen before and after deploying the stent.

In some cases, the stent may include a stent body having a first diameter and a first length in a deployed configuration. The stent may include the helical wrapping pattern that is at least partially covered with a material. In some cases, the stent may include at least two anchoring members coupled with a distal portion and a proximal portion, respectively, of the stent body. For example, the stent may include a first anchoring member coupled with a distal portion of the stent body and configured to increase a diameter of the distal portion of the stent body to a second diameter greater than the first diameter. The stent may further include a second anchoring member coupled with a proximal portion of the stent body and configured to increase a diameter of the proximal portion of the stent body to the second diameter greater than the first diameter. Each of the anchoring members may be configured to anchor the distal portion and proximal portion of the stent within the respective body lumens such that the stent remains in a fixed position.

Upon retraction of the outer sheath, the stent releases from within the outer sheath and expands into a deployed configuration within the body lumen. For example, the distal portion including the first anchoring member may deploy from the outer sheath into a deployed configuration within the first body lumen. In such cases, the first anchoring member of the stent may accurately and precisely deploy in the first body lumen, thereby enabling the stent to be able to form a bridge between two body lumens and bypass the obstruction. For example, the distal portion of the stent (e.g., the first anchoring member) may deploy in the first body lumen and anchor itself within the first body lumen.

In some cases, the proximal portion including the second anchoring member may precisely deploy within the second body lumen and allow the stent to bridge between two body lumens upon expansion. The outer sheath may be withdrawn past a distal end of a marker to expand the proximal portion of the stent. In such cases, the second anchoring member of the stent may expand from within the outer sheath such that upon fully exiting the outer sheath, the proximal portion (e.g., the second anchoring member) expands to a deployed configuration within the second body lumen. The second anchoring member may enable the stent to anchor itself within the second body lumen. The deployed configuration may be an example of the stent fully exiting the outer sheath and expanding between the first body lumen and the second body lumen, thereby providing an alternative route to bypass the obstruction.

Embodiments of the present disclosure are now described in detail with reference to the drawings. As used herein, the term “clinician” refers to a doctor, surgeon, nurse, or any other care provider and may include support personnel. The term “proximal” will refer to the portion of the device or component thereof that is closer to the clinician and the term “distal” will refer to the portion of the device or component thereof that is farther from the clinician.

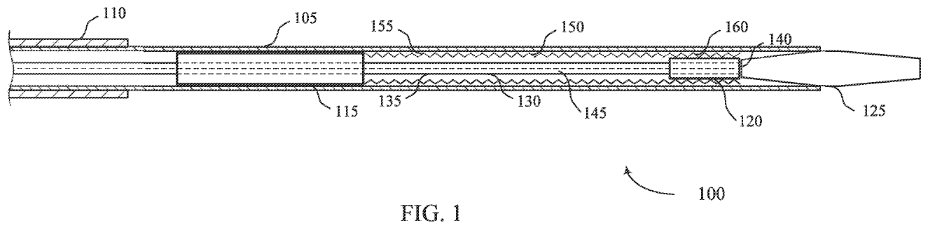

FIG. 1 illustrates a system 100 for providing access to a body lumen and delivering a stent in accordance with aspects of the present disclosure. The system 100 generally includes an outer sheath 105, an isolation sheath 110, a marker 115, an anchoring component 120, an electrocautery tip 125, an inner tubular member 130, a stent 150, and a guidewire 145. The system 100 can be provided as individual components, selectively combined components, or all together as a kit of components.

During a luminal access and stent 150 delivery procedure, the electrocautery tip 125 may access the target body lumen by piercing a wall of the body lumen, for example, to deliver a stent 150. In general, a stent 150 is a frame or scaffolding structure sized for placement within a body lumen and configured to provide structural support to the inner surface of the body lumen. A stent 150 may be used to restore patency across narrowed or blocked areas within the body lumen due to inflammation, tumors, plaque buildup, or any other obstructive feature. Although references to the pancreaticobiliary system are provided herein, it should be appreciated that the stents described herein may be used in any body lumen. Furthermore, as discussed in more detail below, the stent 150 may be disposed around the inner tubular member 130.

The stent 150 may be made from any number of materials, combinations of materials, and constructions. In some examples, the stent 150 is a self-expanding stent. The stent 150 may be a wire-form stent formed by one or more helically wrapped wires. However, it should be appreciated that the stent 150 may be made from other stent constructions or combinations of stent constructions. In other examples, the stent 150 is a laser-cut stent formed from a single metallic tube with regions cut away for increased flexibility. In yet other examples, the stent 150 is a braided stent made from a plurality of wires joined together in a cross-hatch configuration. In some examples, the stent 150 may be a combination of the braided stent and the wire-form stent.

It may be appreciated that the different stent constructions may exhibit particular characteristics such as radial expansive force, flexibility, reduced foreshortening, or migration resistance that may render a certain construction advantageous for a particular use. For example, the helical wrapping pattern of the stent 150 may be configured to reduce a foreshortening of the stent body upon deployment from an undeployed configuration to the deployed configuration to less than ten percent of a length of the stent body in the undeployed configuration. In such cases, the stent 150 may be an example of a non-foreshortening stent.

The individual wires or frame of the stent 150 may be made from any number of metallic materials including, but not limited to, titanium, nitinol, or stainless steel. It should be appreciated that other metallic or non-metallic materials may be used to construct the stent 150 that provides suitable flexibility, stiffness, and biocompatibility. The stent 150 may include a polymeric or fabric sleeve (e.g., first material) that covers some or all of the surface of the stent 150. Such a sleeve may protect the inner surface of the body lumen from the bare metal of the stent 150 and may prevent tissue ingrowth. For example, the stent 150 may include a helical wrapping pattern that is at least partially covered with a first material. In some examples, the stent 150 is a drug-eluting stent.

The outer sheath 105 of the system 100 has an elongate tubular body and an internal lumen extending from its proximal end to the distal end. In general, the outer sheath 105 may be configured to access a body lumen and to provide a conduit through which one or more devices (e.g., a guidewire 145) may pass to facilitate subsequent treatment of the body lumen or associate organs. The outer sheath 105 may include features that facilitate the direction-controlled delivery of a guidewire 145 within the body lumen for subsequent delivery of a stent 150, a biopsy device, a medicinal delivery element, or any number of other treatment or diagnostic devices.

The outer sheath 105 may be disposed coaxially along at least a portion of the inner tubular member 130 such that the stent 150 is disposed between the inner tubular member 130 and the outer sheath 105 while the stent 150 is in an undeployed configuration. The undeployed configuration may be an example of a stent 150 constrained within the outer sheath 105, an unexpanded configuration of the stent 150, or both.

In some examples, the outer sheath 105 may include a lubrication coating disposed within an inner surface of the outer sheath 105. The lubrication coating may be made from a variety of materials, including but not limited to silicone. In such cases, the lubrication coating may reduce deployment forces by at least thirty percent to ensure accurate placement of the stent 150 within the body lumen. In some cases, the lubrication coating of the outer sheath 105 may reduce the friction between the outer sheath 105 and the stent 150 as the outer sheath 105 is retracted over the stent 150. The isolation sheath 110 may be configured to receive the outer sheath 105 as the outer sheath 105 is retracted.

The inner tubular member 130 is generally an elongate, tubular member with a proximal end 135 and distal end 140 and is dimensioned to be advanced through the internal lumen of the outer sheath 105. The inner tubular member 130 may be configured to advance through an access site in a wall of the body lumen. The inner tubular member 130 may be coupled with an anchoring component 120 at the distal end 140 of the inner tubular member 130. In certain embodiments, the distal end 140 of the inner tubular member 130 includes a tip or bulged portion (e.g., anchoring component 120).

The stent 150 may be coupled to the inner tubular member 130 and the anchoring component 120. For example, the stent 150 may be concentric with the inner tubular member 130 and the anchoring component 120. As such, the inner tubular member 130 may extend through the lumen of the stent 150. For example, the stent 150 may be disposed coaxially onto the inner tubular member 130. The stent 150 may be positioned between the outer sheath 105 and the inner tubular member 130 at the proximal end 135 of the inner tubular member 130.

The anchoring component 120 may extend through the lumen of the stent 150 at a distal portion 160 of the stent 150. In such cases, the stent 150 may be positioned between the outer sheath 105 and the anchoring component 120 at the distal end 140 of the inner tubular member 130. For example, the distal portion 160 of the stent 150 may be disposed coaxially along the anchoring component 120 such that the distal portion 160 of the stent 150 is disposed between the anchoring component 120 and the outer sheath 105 while the stent 150 is in the undeployed configuration.

The anchoring component 120 may be made from a variety of materials, including but not limited to silicone. The anchoring component 120 may be disposed at a distal end 140 of the inner tubular member 130 and configured to retain a distal portion 160 of the stent 150 in place along the inner tubular member 130 as the outer sheath 105 is retracted proximally to deploy the stent 150. Upon retraction of the outer sheath 105, the stent 150 may release from the anchoring component 120 and expand into a deployed configuration within the body lumen. The deployed configuration may be an example of an unconstrained configuration, an expanded configuration, or both. The anchoring component 120 may be an example of a bump, an increased diameter component of the inner tubular member 130, a hook, or a combination thereof. In such cases, the anchoring component 120 may be configured to keep the distal portion 160 of the stent 150 stationary as the outer sheath 105 is retracted.

The system 100 may further include a marker 115. The marker 115 may be an example of a proximal marker that is disposed around the inner tubular member 130 and positioned such that a proximal end of the stent 150 abuts against the marker 115 while the stent 150 is in the undeployed configuration. The marker 115 may be configured to indicate a location of the proximal end of the stent 150 within the system 100. The marker 115 may be configured to retain a proximal portion 155 of the stent 150 in place along the inner tubular member 130 as the outer sheath 105 is retracted proximally to deploy the stent 150. In some cases, the marker 115 may be coupled with the inner tubular member 130 such that the marker 115 remains stationary as the outer sheath 105 is retracted. In other examples, the marker 115 is slidably disposed onto the inner tubular member 130 such that the marker 115 is a moving marker as the outer sheath 105 is retracted back to deploy the stent 150.

The marker 115 includes generally an elongate, tubular member and is configured to house the inner tubular member 130. In some cases, the marker 115 may be tapered such that a distal end of the marker 115 may extend underneath the proximal portion 155 of the stent 150. For example, the marker 115 may be an example of a proximal anchoring component such that the marker 115 may be configured to compress the proximal portion 155 of the stent 150 between the marker 115 and the outer sheath 105.

The electrocautery tip 125 may be an example of a distal cutting element coupled with the distal end 140 of the inner tubular member 130 and configured to create the access site in the wall of the body lumen. The electrocautery tip 125 may include a coiled electrode wire that extends radially around a circumference of a distal end of the electrocautery tip 125, a single electrode wire that extends longitudinally and in a proximal direction from a distal end of the electrocautery tip 125, a single, spiral electrode wire that extends around a distal end of the electrocautery tip 125, or an electrode tube. In some case, the electrocautery tip 125 may include a tapered cover disposed around the electrocautery tip 125. The outer sheath 105 may at least partially overlap the tapered cover.

The guidewire 145 is generally a flexible elongate member configured to slidably advance through the internal lumen of the inner tubular member 130. In such cases, the guidewire 145 may be disposed within the inner tubular member 130. The guidewire 145 may be uniform in size and stiffness along its entire length, or alternatively, may include sections of differing stiffness.

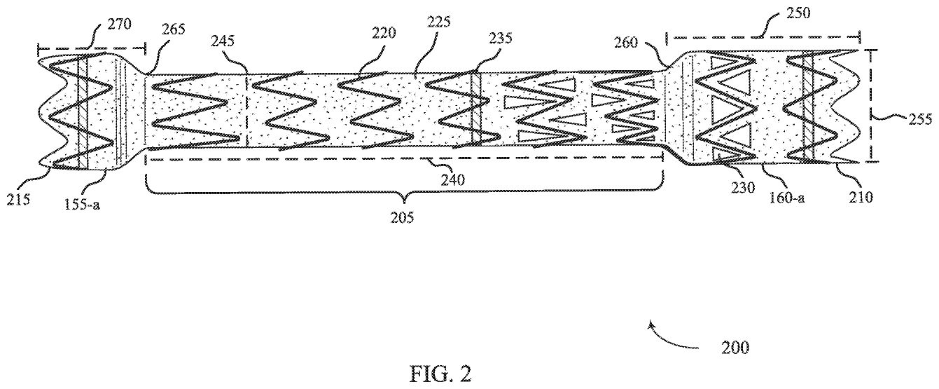

FIG. 2 illustrates a stent 200 with flared portions in accordance with aspects of the present disclosure. The stent 200 may be configured to restore luminal flow across narrowed areas or blockages within a body lumen, as described with reference to FIG. 1. The stent 200 may be sized or otherwise adapted to be placed within any body lumen, such as those associated with the pancreaticobiliary system, the arterial system, the bronchial system, the urinary system, or any other luminal system that may require stent treatment. In some cases, the stent 200 may be placed within the body lumen by a stent delivery system, as described with reference to FIG. 1. The stent 200 may be an example of stent 150 as described with reference to FIG. 1.

The stent 200 may be categorized as having a proximal portion 155-a, which may, for example, be placed within a duodenum, and a distal portion 160-a which may, for example, be placed within a biliary duct. The stent 200 may include a stent body 205 that has a diameter 245 and a length 240 in a deployed configuration. The stent body 205 may extend between the distal portion 160-a and the proximal portion 155-a. The stent body 205 may include a distal end 260 and a proximal end 265. In such cases, the stent body 205 may extend between the distal end 260 and the proximal end 265. The stent body 205 may be an example of a mid-body portion of the stent 200 that includes a narrow region between a first flared portion 210 and a second flared portion 215.

The stent 200 may include a first anchoring member (e.g., first flared portion 210) coupled with a distal end 260 of the stent body 205. The first flared portion 210 may be configured to increase a diameter of the distal portion 160-a of the stent body 205 to a second diameter 255 greater than the first diameter 245. In such cases, the diameter 255 of the first flared portion 210 may be greater than a diameter 245 of the stent body 205 in the deployed configuration. The first flared portion 210 may be coupled with a distal end 260 of the stent body 205 and spaced around a circumference of the distal end 260 of the stent body 205.

The stent 200 may include a second anchoring member (e.g., a second flared portion 215) coupled with a proximal portion 155-a of the stent body 205. The second flared portion 215 may be configured to increase a diameter of the proximal portion 155-a of the stent body 205 to a second diameter 255 greater than the first diameter 245. In such cases, the diameter 255 of the second flared portion 215 may be greater than a diameter 245 of the stent body 205 in the deployed configuration. The second flared portion 215 may be coupled with a proximal end 265 of the stent body 205 and spaced around a circumference of the proximal end 265 of the stent body 205.

In some cases, a length 250 of the first flared portion 210 may be greater than a length 270 of the second flared portion 215. In other examples, the length 250 of the first flared portion 210 may be equal to a length 270 of the second flared portion 215. For example, the first flared portion 210 may have a length 250 ranging from 3 mm to 15 mm. The second flared portion 215 may have a length 270 ranging from 3 mm to 15 mm. In some examples, the length 270 of the second flared portion 215 may be shorter than the length 250 of the first flared portion 210. The diameter 255 of the first flared portion 210 may be the same as the diameter of the second flared portion 215. In some cases, the diameter 255 of the first flared portion 210 may be different than the diameter of the second flared portion 215. The first flared portion 210 and the second flared portion 215 may include the helical wrapping pattern 220.

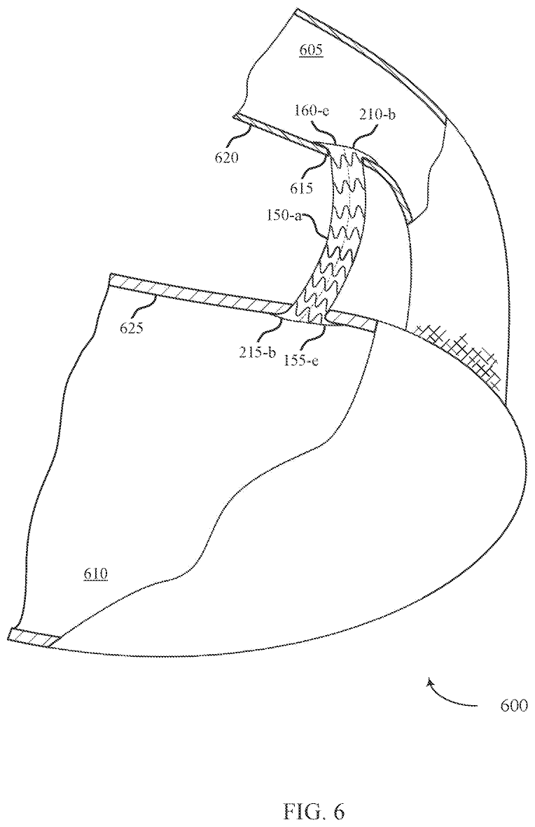

The first flared portion 210 and second flared portion 215 may respectively bridge each end of the stent 200 (e.g., the proximal end 265 and the distal end 260) to the stent body 205. For example, the first flared portion 210 may bridge the stent body 205 with the distal end 260 of the stent 200. The second flared portion 215 may bridge the stent body 205 with the proximal end 265 of the stent 200. In some cases, the transition between the narrower diameter 245 of the stent body 205 and the wider diameters 255 of the first flared portion 210 and the second flared portion 215 may be gradual or steep. The first flared portion 210 and the second flared portion 215 may enable the stent 200 to resist migration within the body lumen by expanding from a undeployed configuration to a deployed configuration, as described with reference to FIG. 6. The stent body 205 may bridge the two body lumens, and the first flared portion 210 and the second flared portion 215 may act as anti-migration tool to prevent the stent 200 from moving further into either body lumen. Using the first flared portion 210 and the second flared portion 215 as an anti-migration tool may be less invasive to the body tissue compared to other anti-migration tools used in stents.

The stent 200 may include a helical wrapping pattern 220 that may be at least partially covered with a cover material 225. The helical wrapping pattern 220 may be configured to reduce a foreshortening of the stent body 205 upon deployment from an undeployed configuration to the deployed configuration to less than ten percent of a length 240 of the stent body 205 in the undeployed configuration. In such cases, the length 240 of the stent body 205 may be maintained before deployment and after deployment to ensure accurate and precise placement with the body lumen. The stent 200 may be an example of a non-foreshortening stent.

The helical wrapping pattern 220 may include a single wire. The single wire of the helical wrapping pattern 220 may be made from any number of metallic materials including, but not limited to, titanium, nitinol, or stainless steel. It should be appreciated that other metallic or non-metallic materials may be used to construct the stent 200 that provide suitable flexibility, stiffness, and biocompatibility. The single wire may be helically wrapped around the stent 200 such that the helical wrapping pattern 220 extends from the proximal portion 155-a and to the distal portion 160-a. In some cases, using the single wire may improve the structural stability of the stent 200 as compared to a multi-wire stent. In some cases, the helical wrapping pattern 220 may enable the stent 200 to evenly withstand pressure across the entire body of the stent 200. The helical wrapping pattern 220 may define at least two diameters of the stent 200 (e.g., diameter 255 of the first flared portion 210 and diameter 245 of the stent body 205). In some cases, the diameter 245 of the stent body 205 may range from 5 mm to 10 mm. The diameter 255 of the first flared portion 210 may range from 8 mm to 14 mm.

In some examples, the cover material 225 may fully cover the stent 200. For example, the cover material 225 may cover an entire portion of the stent body 205, the first flared portion 210, and the second flared portion 215. In some examples, the cover material 225 may at least partially cover the stent body 205, the first flared portion 210, the second flared portion 215, or a combination thereof. The cover material 225 may cover the helical wrapping pattern 220 to protect the body lumen from the metallic contact of the single wire of the helical wrapping pattern 220. In such cases, the cover material 225 may be more biocompatible with the body lumen than the metal of the single wire of the helical wrapping pattern 220. The cover material 225 may be made from any number of materials including, but not limited to, expanded polytetrafluoroethylene-fluoroethylene-propylene (ePTFE-FEP), a plastic material, or silicone. In some cases, the cover material 225 may include one or more layers. For example, the cover material 225 may include layers of ePTFE which may include pores and layers of FEP which may enable the stent 200 to be impermeable to certain pressure levels. In some cases, the cover material 225 may be selected to be thin enough to achieve a smaller profile of the constrained diameter 245 of the stent body 205. For example, the smaller profile of the stent body 205 may enable a more efficient stent delivery.

In some examples, the cover material 225 may include cut-out drainage holes, such as the drainage holes 230. In some cases, the drainage holes 230 may enable fluid drainage into the body lumen, which may increase the efficiency of the stent 200. In some cases, the drainage holes 230 may enable drainage across a duct where stent placement may be desired. In some cases, the placement of the drainage holes 230 at the distal portion 160-a of the stent 200 may enable bile drainage while also preventing food or other debris from traveling up the lumen of the stent 200, thereby causing an occlusion. In some cases, the drainage holes 230 may be disposed within the second flared portion 215 and the proximal portion 155-a of the stent body 205. In such cases, the drainage holes 230 may be interspersed around the circumference of the stent 200 near the proximal end 265. In some cases, one or more drainage holes 230 may be laser cut into the cover material 225. In some cases, the drainage holes 230 may be a triangular shape and located at each point of the helical wrapping pattern 220 of the single wire. In some cases, the drainage holes 230 may be disposed within the proximal portion 155-a, the stent body 205, the distal portion 160-a, or a combination thereof.

The stent 200 may include marker bands 235. The marker bands 235 may be placed at each section of the stent 200. For example, the marker bands 235 may be disposed around the stent body 205, the first flared portion 210, and the second flared portion 215. The marker bands 235 may aid in stent placement, as described with reference to FIGS. 1 and 6. In some cases, the marker bands 235 may be made from any number of metallic materials including, but not limited to, gold. In such cases, the marker bands 235 may enable visual differentiation from the cover material 225 as well as the gold material of the marker bands 235 being more biocompatible with the body lumen than other metals.

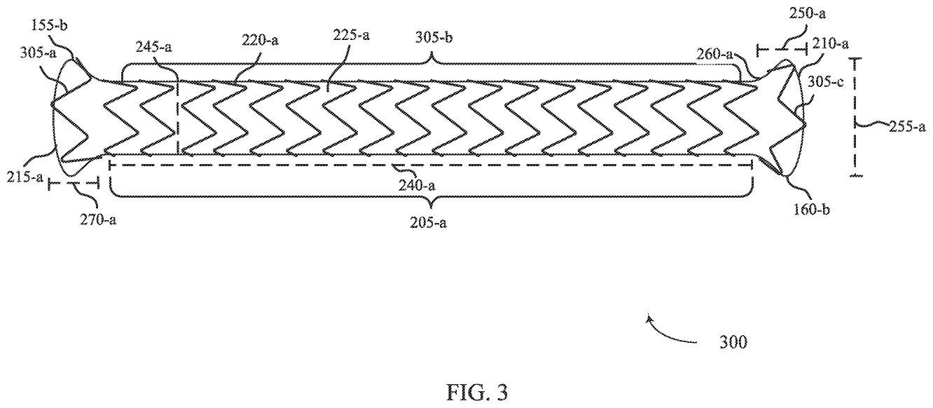

The stent 200 may be made from any number of materials, combinations of materials, and constructions. In some examples, the stent 200 may be a laser-cut stent formed from a single metallic tube with regions cut way for increased flexibility. For example, the helical wrapping pattern 220 may include a laser cut frame. In some examples, the stent 200 may be a wire-formed stent formed by one or more helically wrapped wires, as described with reference to FIG. 3. It may be appreciated that the different stent constructions may exhibit particular characteristics such as radial expansive force, flexibility, reduced foreshortening, or migration resistance that may render a certain construction advantageous for a particular use.

FIG. 3 illustrates a stent 300 with more than one wire frame 305 in accordance with aspects of the present disclosure. The stent 300 may be configured to restore luminal flow across narrowed areas or blockages within a body lumen, as described with reference to FIGS. 1 and 2. The stent 300 may be sized or otherwise adapted to be placed within any body lumen, such as those associated with the pancreaticobiliary system, the arterial system, the bronchial system, the urinary system, or any other luminal system that may require stent treatment. In some cases, the stent 300 may be placed within the body lumen by a stent delivery system, as described with reference to FIG. 1. The stent 300 may be an example of stent 150 as described with reference to FIG. 1.

The stem 300 may be categorized as having a proximal portion 155-b, which may, for example, be placed within a duodenum, and a distal portion 160-b which may, for example, be placed within a biliary duct. The stent 300 may include a stent body 205-b that has a diameter 245-a and a length 240-a in a deployed configuration. The stent body 205-a may extend between the distal portion 160-b and the proximal portion 155-b. The stent body 205-a may include a distal end 260-a and a proximal end 265-a. In such cases, the stent body 205-a may extend between the distal end 260-a and the proximal end 265-a. The stent body 205-a may be an example of a mid-body portion of the stent 300 that includes a narrow region between a first flared portion 210-a and a second flared portion 215-a.

The stent 30) may include a first anchoring member (e.g., first flared portion 210-a) coupled with a distal end 260-a of the stent body 205-a. The first flared portion 210-a may be configured to increase a diameter of the distal portion 160-b of the stent body 205-a to a second diameter 255-a greater than the first diameter 245-a. In such cases, the diameter 255-a of the first flared portion 210-a may be greater than a diameter 245-a of the stent body 205-a in the deployed configuration. The first flared portion 210-a may be coupled with a distal end 260-a of the stent body 205-a and spaced around a circumference of the distal end 260-a of the stent body 205-a.

The stent 300 may include a second anchoring member (e.g., a second flared portion 215-a) coupled with a proximal portion 155-b of the stent body 205-a. The second flared portion 215-a may be configured to increase a diameter of the proximal portion 155-b of the stent body 205-a to a second diameter 255-a greater than the first diameter 245-a. In such cases, the diameter 255-a of the second flared portion 215-a may be greater than a diameter 245-a of the stent body 205-a in the deployed configuration. The second flared portion 215-a may be coupled with a proximal end 265-a of the stent body 205-a and spaced around a circumference of the proximal end 265-a of the stent body 205-a.

In some cases, a length 250-a of the first flared portion 210-a may be equal to a length 270-a of the second flared portion 215-a. The diameter 255-a of the first flared portion 210-a may be the same as the diameter of the second flared portion 215-a. In some cases, the diameter 255-a of the first flared portion 210-a may be different than the diameter of the second flared portion 215-a. The first flared portion 210-a and the second flared portion 215-a may include the helical wrapping pattern 220-a.

The first flared portion 210-a and second flared portion 215-a may respectively bridge each end of the stent 300 (e.g., the proximal end 265-a and the distal end 260-a) to the stent body 205-a. For example, the first flared portion 210-a may bridge the stent body 205-a with the distal end 260-a of the stent 200-a. The second flared portion 215-a may bridge the stent body 205-a with the proximal end 265-a of the stent 200-a. In some cases, the transition between the narrower diameter 245-a of the stent body 205-a and the wider diameters 255-a of the first flared portion 210-a and the second flared portion 215-a may be gradual or steep. The first flared portion 210-a and the second flared portion 215-a may enable the stent 300 to resist migration within the body lumen by expanding from a undeployed configuration to an deployed configuration, as described with reference to FIG. 6. The stent body 205-a may bridge the two body lumens, and the first flared portion 210-a and the second flared portion 215-a may act as anti-migration tool to prevent the stent 200 from moving further into either body lumen. Using the first flared portion 210-a and the second flared portion 215-a as an anti-migration tool may be less invasive to the body tissue compared to other anti-migration tools used in stents.

The helical wrapping pattern 220-a may include more than one wire frame 305. For example, the helical wrapping pattern 220-a may include wire frame 305-a, 305-b, and 305-c. The series of wire frames 305 may extend along the entire length of the stent 300. In some cases, the wire frames 305 may be welded together at one or more points along the stent body 205-a. In some cases, each of the wire frames 305 may be separate from each other wire frame 305. For example, wire frame 305-a may be a separate wire from wire frame 305-b, and wire frame 305-b may be separate from wire frame 305-c. In some cases, more than one wire frame 305 may be grouped together to form a sub-unit of wire frames 305. In such cases, stent 300 may include more than one sub-unit of wire frames 305. In some examples, the first flared portion 210-a may include a wire frame 305-c, the stent body 205-a may include at least a wire frame 305-b, and the second flared portion 215-b may include a wire frame 305-a. In such cases, the stent 300 may include three sub-units of wire frames 305. In some cases, the wire frame 305-b of the stent body 205-a may be disconnected from wire frame 305-a and wire frame 305-c. In such cases, the wire frames 305-a, 305-b, and 305-c may be connected via the cover material 225-a.

The wire frames 305 of the helical wrapping pattern 220-a may be made from any number of metallic materials including, but not limited to, titanium, nitinol, or stainless steel. It should be appreciated that other metallic or non-metallic materials may be used to construct the stent 300 that provide suitable flexibility, stiffness, and biocompatibility. The wire frames 305 may be wrapped around the stent 300 such that the helical wrapping pattern 220-a extends from the proximal portion 155-b and to the distal portion 160-b. The wire frames 305 may define at least two diameters of the stent 300 (e.g., diameter 255-a of the first flared portion 210-a and diameter 245-a of the stent body 205-a).

In some cases, the wire frames 305 may each include different diameters (e.g., thickness) of the wires. For example, the wire frame 305-b along the stent body 205-a may include a diameter different than the wire frame 305-c and wire frame 305-a at the first flared portion 210-a and the second flared portion 215-a, respectively. In some cases, the wire frames 305-a and 305-c (e.g., the wire frames 305 located at each end of the stent 300) may include a diameter larger than the diameter of the wire frame 305-b of the stent body 205-a.

In some examples, the cover material 225-a may fully cover the stent 300. For example, the cover material 225-a may cover an entire portion of the stent body 205-a, the first flared portion 210-a, and the second flared portion 215-a. In some examples, the cover material 225-a may at least partially cover the stent body 205-a, the first flared portion 210-a, the second flared portion 215-a, or a combination thereof. The cover material 225-a may cover the helical wrapping pattern 220-a to protect the body lumen from the metallic contact of the more than one wire frames 305 of the helical wrapping pattern 220-a. In such cases, the cover material 225-a may be more biocompatible with the body lumen than the metal of the more than one wire frame 305 of the helical wrapping pattern 220-a.

The cover material 225-a may be made from any number of materials including, but not limited to, expanded polytetrafluoroethylene-fluoroethylene-propylene (ePTFE-FEP), a plastic material, or silicone. In some cases, the cover material 225-a may be selected to be thin enough to achieve a smaller profile of the constrained diameter 245-a of the stent body 205-a. For example, the smaller profile of the stent body 205-a may enable a more efficient stent delivery. In some cases, the cover material 225-a may provide the connective material between adjacent wire frames 305. For example, the overall structure of the stent 300 may be provided by the combination of the wire frames 305 and the cover material 225-a. In some cases, the stent 300 may include drainage holes.

The stent 300 may be made from any number of materials, combinations of materials, and constructions. It may be appreciated that the different stent constructions may exhibit particular characteristics such as radial expansive force, flexibility, reduced foreshortening, or migration resistance that may render a certain construction advantageous for a particular use.

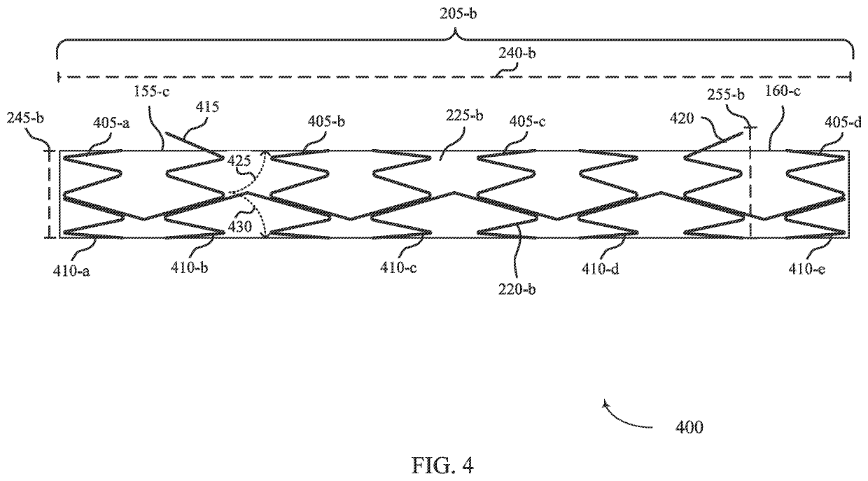

FIG. 4 illustrates a stent 400 with anchoring fins in accordance with aspects of the present disclosure. The stent 400 may be configured to restore luminal flow across narrowed areas or blockages within a body lumen, as described with reference to FIGS. 1-3. The stent 400 may be sized or otherwise adapted to be placed within any body lumen, such as those associated with the pancreaticobiliary system, the arterial system, the bronchial system, the urinary system, or any other luminal system that may require stent treatment. In some cases, the stent 400 may be placed within the body lumen by a stent delivery system, as described with reference to FIG. 1. The stent 400 may be an example of stent 150 as described with reference to FIG. 1.

The stent 400 may be categorized as having a proximal portion 155c, which may, for example, be placed within a duodenum, and a distal portion 160-c which may, for example, be placed within a biliary duct. The stent 400 may include a stent body 205-b that has a diameter 245-b and a length 240-b in a deployed configuration. The stent body 205-b may extend between the distal portion 160-c and the proximal portion 155-c.

The stent 400 may include a first anchoring member (e.g., first fin 420) coupled with a distal portion 160-c of the stent 400. The first fin 420 may protrude from the distal portion 160-c of the stent body 205-b and extend in a distal direction. In some cases, the first fin 420 may extend in a proximal direction. The first fin 420 may be configured to increase a diameter of the distal portion 160-c of the stent body 205-b to a second diameter 255-b greater than the first diameter 245-b. In such cases, the diameter 255-b of the first fin 420 may be greater than a diameter 245-b of the stent body 205-b in the deployed configuration.

The stent 400 may include a second anchoring member (e.g., second fin 415) coupled with a proximal portion 155-c of the stent 400. The second fin 415 may protrude from the proximal portion 155-c of the stent body 205-b and extend in a proximal direction. In some cases, the second fin 415 may extend in a distal direction. The second fin 415 may be configured to increase a diameter of the proximal portion 155-c of the stent body 205-b to a second diameter 255-b greater than the first diameter 245-b. In such cases, the diameter 255-b of the second fin 415 may be greater than a diameter 245-b of the stent body 205-b in the deployed configuration. In some cases, a distance that the second fin 415 extends from the surface of the stent body 205-b may be different than a distance that the first fin 420 extends from the surface of the stent body 205-b. In some examples, the distance that the second fin 415 extends from the surface of the stent body 205-b may be the same as a distance that the first fin 420 extends from the surface of the stent body 205-b.

The first fin 420 and the second fin 415 may enable the stent 400 to resist migration within the body lumen by expanding from a undeployed configuration to an deployed configuration, as described with reference to FIG. 6. The stent body 205-b may bridge the two body lumens, and the first fin 420 and the second fin 415 may act as anti-migration tool to prevent the stent 400 from moving further into either body lumen. Using the first fin 420 and the second fin 415 as an anti-migration tool may be less invasive to the body tissue compared to other anti-migration tools used in stents. In some cases, compared with the anchoring members as described with reference FIGS. 2 and 3, the first fin 420 and the second fin 415 may allow for a smaller constrained profile of the stent 400 when the stent 400 is loaded in the delivery system, as described with reference to FIG. 1.

The stent 400 may include a helical wrapping pattern 220-b that may be at least partially covered with a cover material 225-b. The helical wrapping pattern 220-b may be configured to reduce a foreshortening of the stent body 205-b upon deployment from an undeployed configuration to the deployed configuration to less than ten percent of a length 240-b of the stent body 205-b in the undeployed configuration. In such cases, the length 240-b of the stent body 205-b may be maintained before deployment and after deployment to ensure accurate and precise placement with the body lumen. The stent 400 may be an example of a non-foreshortening sten.

The helical wrapping pattern 220-b may include first wire frames 405 and second wire frames 410. The first wire frames 405 may include at least first wire frames 405-a, 405-b, 405-c, and 405-d. The first wire frames 405 may wrap around a first portion 425 of the circumference of the stent body 205-b. For example, the first portion 425 of the circumference of the stent body 205-b may include a top half (e.g., hemisphere) of the stent body 205-b that extends from a midpoint on a first side of the stent body 205-b, over the top of the stent body 205-b, and to a midpoint on a second side of the stent body 205-b. In such case, the first wire frames 405 include half rings that form half of the circumference of the stent 400. In some cases, the first wire frame 405-a may include the second fin 415, and the first wire frame 405-d may include the first fin 420.

The first fin 420 may be formed by bending and/or shape setting a crown of the helical wrapping pattern 220-b. For example, a crown (e.g., point) of the first wire frame 405-d may be bent out of a plane of the stent body 205-b such that the angle of the first fin 420 may range from 30 degrees to 55 degrees relative to the plane parallel to a central axis of the stent body 205-b. In some examples, the second fin 415 may be formed by bending and/or shape setting a crown of the helical wrapping pattern 220-b. For example, a crown (e.g., point) of the first wire frame 405-a may be bent out of a plane of the stent body 205-b such that the angle of the second fin 415 may range from 30 degrees to 55 degrees relative to the plane parallel to a central axis of the stent body 205-b.

The second wire frames 410 may include at least second wire frames 410-a. 410-b, 410-c, 410-d, and 410-e. The second wire frames 410 may wrap around a second portion 430 of the circumference of the stent body 205-b. For example, the second portion 430 of the circumference of the stent body 205-b may include a bottom half (e.g., hemisphere) of the stent body 205-b that extends from the midpoint on the first side of the stent body 205-b, over the bottom of the stent body 205-b, and to the midpoint on the second side of the stent body 205-b. In such case, the second wire frames 410 include half rings that form half of the circumference of the stent 400. In some cases, the second wire frames 410 may include the first fin 420 and the second fin 415. In some examples, the first wire frame 405 may include the first fin 420, and the second wire frame 410 may include the second fin 415, or vice versa.

The first wire frames 405 and the second wire frames 410 may be connected across the length 240-b of the stent body 205-b. For example, the first wire frame 405-a may be connected (e.g., welded) to both the second wire frame 410-a and the second wire frame 410-b. The second wire frame 410-b may be connected to both the first wire frame 405-a and the first wire frame 405-b. By connecting first wire frames 405 and second wire frames 410 in a staggered fashion along the length 240-b of the stent body 205-b (e.g., in a zig-zag pattern), the stent 400 may have improved structural integrity while maintaining flexibility. The welding of first wire frames 405 and second wire frames 410 may also contribute to increased axial stiffness of the stent 400, which may help prevent compression during deployment and thereby improve deployment accuracy.

The first wire frames 405 and the second wire frames 410 of the helical wrapping pattern 220-b may be made from any number of metallic materials including, but not limited to, titanium, nitinol, or stainless steel. It should be appreciated that other metallic or non-metallic materials may be used to construct the stent 400 that provide suitable flexibility, stiffness, and biocompatibility. The first wire frames 405 and the second wire frames 410 may be helically wrapped around the stent 400 such that the helical wrapping pattern 220-b extends from the proximal portion 155-c and to the distal portion 160-c. In some cases, the helical wrapping pattern 220-b may enable the stent 400 to evenly withstand pressure across the entire body of the stent 400.

In some cases, the cover material 225-b may be disposed onto an entire portion of the stent body 205-b, a first fin 420 (e.g., a first anchoring member), and a second fin 415 (e.g., a second anchoring member). In such cases, the cover material 225-b may fully cover the stent 400. The cover material 225-b may cover the helical wrapping pattern 220-b to protect the body lumen from the metallic contact of the first wire frames 405 and the second wire frames 410 of the helical wrapping pattern 220-b. In such cases, the cover material 225-b may be more biocompatible with the body lumen than the metal of the first wire frames 405 and the second wire frames 410 of the helical wrapping pattern 220-b. The cover material 225-b may be made from any number of materials including, but not limited to, expanded polytetrafluoroethylene-fluoroethylene-propylene (ePTFE-FEP), a plastic material, or silicone. In some cases, the cover material 225-b may be selected to be thin enough to achieve a smaller profile of the constrained diameter 245-b of the stent body 205-b. For example, the smaller profile of the stent body 205-b may enable a more efficient stent delivery.

The stent 400 may be made from any number of materials, combinations of materials, and constructions. In some examples, the stent 400 may be a laser-cut stent formed from a single metallic tube with regions cut away for increased flexibility. For example, the helical wrapping pattern 220-b may include one or more laser-cut frames. In some examples, each of the first wire frames 405 and each of the second wire frames 410 may be an example of a laser-cut frame. It may be appreciated that the different stent constructions may exhibit particular characteristics such as radial expansive force, flexibility, reduced foreshortening, or migration resistance that may render a certain construction advantageous for a particular use.

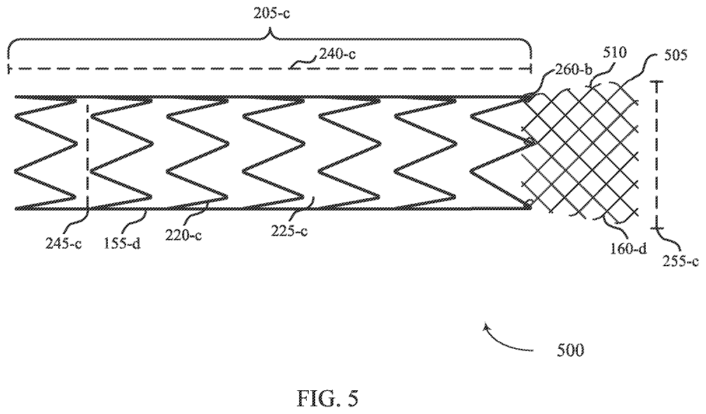

FIG. 5 illustrates a hybrid stent 500 in accordance with aspects of the present disclosure. The stent 500 may be configured to restore luminal flow across narrowed areas or blockages within a body lumen, as described with reference to FIGS. 1-4. The stent 500 may be sized or otherwise adapted to be placed within any body lumen, such as those associated with the pancreaticobiliary system, the arterial system, the bronchial system, the urinary system, or any other luminal system that may require stent treatment. In some cases, the stent 500 may be placed within the body lumen by a stent delivery system, as described with reference to FIG. 1. The stent 500 may be an example of stent 150 as described with reference to FIG. 1.

The stent 500 may be categorized as having a proximal portion 155-d, which may, for example, be placed within a duodenum, and a distal portion 160-d which may, for example, be placed within a biliary duct. The stent 500 may include a stent body 205-c that has a diameter 245-c and a length 240c in a deployed configuration. The stent body 205-c may extend between the distal portion 160-d and the proximal portion 155-d. The stent body 205-c may include a distal end 260-b. The stent body 205-c may be an example of a mid-body portion of the stent 500 that includes a narrow region as compared to a flared portion 510.

The stent 500 may include a first anchoring member (e.g., flared portion 510) coupled with a distal end 260-b of the stent body 205-c. The flared portion 510 may be configured to increase a diameter of the distal portion 160-d of the stent body 205-c to a second diameter 255-c greater than the first diameter 245-c. In such cases, the diameter 255-c of the flared portion 510 may be greater than a diameter 245-c of the stent body 205-c in the deployed configuration. The flared portion 510 may be coupled with a distal end 260-b of the stent body 205-b and spaced around a circumference of the distal end 260-b of the stent body 205-c.

The flared portion 510 and the proximal portion 155-d may respectively bridge each end of the stent 500 (e.g., the proximal end and the distal end 260-b) to the stent body 205-c. For example, the flared portion 510 may bridge the stent body 205-c with the distal end 260-b of the stent 500. In some cases, the transition between the narrower diameter 245-c of the stent body 205-c and the wider diameter 255-c of the flared portion 510 may be gradual or steep. The flared portion 510 may enable the stent 50M) to resist migration within the body lumen by expanding from a undeployed configuration to an deployed configuration, as described with reference to FIG. 6. The stent body 205-c may bridge the two body lumens, and the flared portion 510 may act as anti-migration tool to prevent the stent 500 from moving further into either body lumen. Using the flared portion 510 as an anti-migration tool may be less invasive to the body tissue compared to other anti-migration tools used in stents.

The stent 500 may include a helical wrapping pattern 220-c that may be at least partially covered with a cover material 225-c. The helical wrapping pattern 220-c may be configured to reduce a foreshortening of the stent body 205-c upon deployment from an undeployed configuration to the deployed configuration to less than ten percent of a length 240-c of the stent body 205-c in the undeployed configuration. In such cases, the length 240-c of the stent body 205-c may be maintained before deployment and after deployment to ensure accurate and precise placement with the body lumen. The portion of the stent 500 with the helical wrapping pattern 220-c may be an example of a non-foreshortening stent.

The helical wrapping pattern 220-c may include a single wire. The single wire of the helical wrapping pattern 220-c may be made from any number of metallic materials including, but not limited to, titanium, nitinol, or stainless steel. It should be appreciated that other metallic or non-metallic materials may be used to construct the stent 500 that provide suitable flexibility, stiffness, and biocompatibility. The single wire may be helically wrapped around the stent 500 such that the helical wrapping pattern 220-c extends from the proximal portion 155-c and to the distal end 260-b. In some cases, using the single wire may improve the structural stability of the stent 500 as compared to a multi-wire stent. In some cases, the helical wrapping pattern 220-c may enable the stent 500 to evenly withstand pressure across the entire body of the stent 500.

In some examples, the cover material 225-c may cover the stent body 205-c that includes the helical wrapping pattern 220-c. For example, the cover material 225-c may cover an entire portion of the stent body 205-c. In some examples, the cover material 225-c may at least partially cover the stent body 205-c. The cover material 225-c may cover the helical wrapping pattern 220-c to protect the body lumen from the metallic contact of the single wire of the helical wrapping pattern 220-c. In such cases, the cover material 225-c may be more biocompatible with the body lumen than the metal of the single wire of the helical wrapping pattern 220-c. The cover material 225-c may be made from any number of materials including, but not limited to, expanded polytetrafluoroethylene-fluoroethylene-propylene (ePTFE-FEP), a plastic material, or silicone. In some cases, the cover material 225-c may be selected to be thin enough to achieve a smaller profile of the constrained diameter 245-c of the stent body 205-c. For example, the smaller profile of the stent body 205-c may enable a more efficient stent delivery.

The stent 500 may include a braided wrapping pattern 505. The braided wrapping pattern 505 may be coupled with the distal end 260-b of the stent body 205-c. In such cases, the braided wrapping pattern 505 may be located at the distal portion 160-d of the stent 500. The braided wrapping pattern 505 may include a braided frame. The braided frame may be made from a plurality of wires joined together in a cross-hatch configuration. The braided frame of the braided wrapping pattern 505 may be made from any number of metallic materials including, but not limited to, titanium, nitinol, or stainless steel. In some cases, the helical wrapping pattern 220-c and the braided wrapping pattern 505 may be made from the same material or different materials.

The braided wrapping pattern 505 may be uncovered from the cover material 225-c. In some cases, the entire stent 500 may uncovered by the cover material 225-c. The stent 500 may be categorized as a hybrid design (e.g., including both the helical wrapping pattern 220-c and the braided wrapping pattern 505). In some examples, the braided wrapping pattern 505 may be connected to the helical wrapping pattern 220-c via one or more wires looped through the ends of the helical wrapping pattern 220-c at the distal end 260-b.

The braided wrapping pattern 505 include the flared portion 510. In some examples, the flared portion 510 of the braided wrapping pattern 505 may include a 90 degree flare, a 45 degree flare, or a star-shaped flare. In some examples, the braided wrapping pattern 505 may include one or more fins as additional anti-migration tools, as described with reference to FIG. 4.

The stent 500 may be made from any number of materials, combinations of materials, and constructions. It may be appreciated that the different stent constructions may exhibit particular characteristics such as radial expansive force, flexibility, reduced foreshortening, or migration resistance that may render a certain construction advantageous for a particular use.

FIG. 6 illustrates a stent delivery system 600 with the stent 150-a fully deployed in accordance with aspects of the present disclosure. The stent delivery system 600 may generally include the stent 150-a, which may be examples of the corresponding stents described with reference to FIGS. 1 through 5.

The stent delivery system 600 may be configured to place a stent 150-a within a first body lumen 605 to restore luminal flow from a first body lumen 605 to a second body lumen 610, thereby bypassing narrowed areas or blockages within at least the first body lumen 605. The stent delivery system 600 may be sized or otherwise adapted to place a stent within any body lumen, such as those associated with the pancreaticobiliary system, the arterial system, the bronchial system, the urinary system, or any other luminal system that may require stent treatment.

The illustrated portions of the system include the first body lumen 605 (e.g., a common bile duct), which drains bile from both the cystic duct (which drains from the gallbladder) and the common hepatic duct (which drains from the liver) into the second body lumen 610 (e.g., duodenum), where the bile mixes and reacts with digesting food. A clinician may advance an endoscope (e.g., an EUS endoscope) into the lumen of a patient's duodenum (e.g., second body lumen 610) to a position in which the bile ducts may be visualized (e.g., via endosonography). The clinician may then access the common bile duct (e.g., first body lumen 605) by advancing a separate access device from a working channel of the endoscope, through the wall 625 of the duodenum (i.e., trans-duodenally), and then through the wall 620 of the common bile duct.

The stent delivery system 600 may be configured for choledochoduodenostomy (CDS) and hepaticogastrostomy (HGS) procedures in which the stent 150-a is implanted across two tissues layers (e.g., duodenum to common bile duct or stomach to intrahepatic duct). In some cases, the stent delivery system 600 may be configured for transmittal biliary drainage. In such cases, the stent 150-a may bridge between the second body lumen 610 (e.g., the duodenum) and a portion of the first body lumen 605 (e.g., the biliary duct) to create a bridge to bypass an obstruction. The obstruction may be an example of a distal malignant biliary obstruction that obstructs drainage. For example, the stent delivery system 600 may be configured to provide access to at least the common biliary duct to facilitate subsequent procedures to treat narrowed areas or blockages within the bile duct and create a bypass around the narrowed areas or blockages within the bile duct to provide access to the stomach and from the biliary duct via the stent 150-a.

The distal portion 160-e of the stent 150-a may include the first flared portion 210-b. In such cases, the first flared portion 210-b may expand within the first body lumen 605 in direct response to retracting the outer sheath (not shown) past the anchoring component (not shown). As the outer sheath is removed through the access site 615, the distal portion 160-e of the stent 150-a expands to expose the first flared portion 210-b. As the distal portion 160-e of the stent 150-a expands, the first flared portion 210-b contacts the wall 620 of the first body lumen 605. The first flared portion 210-b (e.g., distal portion 160-e) of the stent 150-a may be anchored within the first body lumen 605 such that the distal portion 160-e of the stent 150-a remains in a fixed position. In that case, the distal portion 160-c prevents the stent 150-a from being further withdrawn through the access site 615. The clinician may be able to feel the resistance of the first flared portion 210-b against the first body lumen 605 and may therefore infer the location of the stent 150-a. Additionally or alternatively, the distal portion 160-e of the stent 150-a may be viewed under fluoroscopy or similar imaging techniques to infer the location of the stent 150-a.

The distal portion 160-c of the stent 150-a may deploy in a same location as compared to a location prior to retracting the outer sheath. In the case of a non-foreshortening stent, the stent 150-a may deploy at a same location that the stent 150-a is placed within the first body lumen 605. Once the outer sheath is retracted, the distal portion 160-c of the stent 150-a may expand into the first body lumen 605 and anchor itself to the first body lumen 605. In such cases, the anchored distal portion 160-c of the stent 150-a may maintain the stent in a stationary position as the outer sheath is retracted.

To deploy the stent 150-a within the first body lumen 605 and second body lumen 610, the outer sheath may be retracted past a distal end of the marker (not shown). In the case of a non-foreshortening stent, the stent 150-a expands to contact the inner surface of the first body lumen 605 and the inner surface of the second body lumen 610 such that the stent 150-a forms a bridge between the first body lumen 605 and the second body lumen 610.

The proximal portion 155-c of the stent 150-a may expand from within the outer sheath such that upon fully exiting the outer sheath, the proximal portion 155-e expands to a deployed configuration within the second body lumen 610. In such cases, the entire portion of the stent 150-a may expand such that at least a portion of the stent 150-a extends through the first body lumen 605 and into the second body lumen 610.

The outer sheath may be retracted to a second position (e.g., past the distal end of the marker), and the proximal portion 155-e of the stent 150-a may deploy in a same location as compared to a location prior to retracting the outer sheath. In the case of a non-foreshortening stent, the stent 150-a may deploy at a same location that the stent 150-a is placed within the second body lumen 610. Once the outer sheath is retracted, the proximal portion 155-e of the stent 150-a may expand into the second body lumen 610 and anchor itself to the second body lumen 610. In such cases, the anchored proximal portion 155-e of the stent 150-a may maintain the stent 150-a in a stationary position.

The proximal portion 155-e of the stent 150-a may include a second flared portion 215-b. In such cases, the second flared portion 215-b may expand within the second body lumen 610 in direct response to retracting the outer sheath. The proximal portion 155-e of the stent 150-a may expand to expose the second flared portion 215-b. As the proximal portion 155-e of the stent 150-a expands, the second flared portion 215-b contacts the wall 625 of the second body lumen 610. The second flared portion 215-b (e.g., proximal portion 155-e) of the stent 150-a may be anchored within the second body lumen 610 such that the proximal portion 155-c of the stent 150-a remains in a fixed position.

The first flared portion 210-b (e.g., distal portion 160-e) of the stent 150-a may be anchored within the first body lumen 605 such that the distal portion 160-e of the stent 150-a remains in a fixed position. In such cases, the distal portion 160-c of the stent 150-a may be compressed against the wall 620 of the first body lumen 605 after deploying the distal portion 160-e of the stent 150-a from the outer sheath. Furthermore, the stent 150-a may at least partially cover the access site 9615.

The second flared portion 215-b (e.g., proximal portion 155-e) of the stent 150-a may be anchored within the second body lumen 610 such that the proximal portion 155-c of the stent 150-a remains in a fixed position. In such cases, the proximal portion 155-c of the stent 150-a may be compressed against the wall 625 of the second body lumen 610 after expanding the proximal portion 155-e of the stent 150-a from within the outer sheath. Furthermore, the stent 150-a may at least partially cover the access site of the second body lumen 610.

It should be noted that these methods describe possible implementation, and that the operations and the steps may be rearranged or otherwise modified such that other implementations are possible. In some examples, aspects from two or more of the methods may be combined. For example, aspects of each of the methods may include steps or aspects of the other methods, or other steps or techniques described herein.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not to be limited to the examples and designs described herein but is to be accorded the broadest scope consistent with the principles and novel features disclosed herein.

While several embodiments of the present disclosure have been described and illustrated herein, those of ordinary skill in the art will readily envision a variety of other means or structures for performing the functions or obtaining the results or one or more of the advantages described herein, and each of such variations or modifications is deemed to be within the scope of the present disclosure. More generally, those skilled in the art will readily appreciate that all parameters, dimensions, materials, and configurations described herein are meant to be exemplary and that the actual parameters, dimensions, materials, or configurations will depend upon the specific application or applications for which the teachings of the present disclosure is/are used.

Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, or method described herein. In addition, any combination of two or more such features, systems, articles, materials, kits, or methods, if such features, systems, articles, materials, kits, or methods are not mutually inconsistent, is included within the scope of the present disclosure.

All definitions, as defined and used herein, should be understood to control over dictionary definitions, definitions in documents incorporated by reference, or ordinary meanings of the defined terms.

The indefinite articles “a” and “an,” as used herein in the specification and in the claims, unless clearly indicated to the contrary, should be understood to mean “at least one.” Also, as used herein, including in the claims, “or” as used in a list of items (for example, a list of items prefaced by a phrase such as “at least one of” or “one or more”) indicates an inclusive list such that, for example, a list of at least one of A, B, or C means A or B or C or AB or AC or BC or ABC (i.e., A and B and C).

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

What is claimed is:

1. A stent comprising:
a stent body having a first diameter in a deployed configuration, a first length in the deployed configuration, and comprising a helical wrapping pattern that is at least partially covered with a first material, wherein the helical wrapping pattern is configured to reduce a foreshortening of the stent body upon deployment from an undeployed configuration to the deployed configuration to less than ten percent of a length of the stent body in the undeployed configuration;
a first anchoring member coupled with a distal portion of the stent body and configured to increase a diameter of the distal portion of the stent body to a second diameter greater than the first diameter, and
a second anchoring member coupled with a proximal portion of the stent body and configured to increase a diameter of the proximal portion of the stent body to the second diameter greater than the first diameter.

2. The stent of claim 1, wherein the first anchoring member comprises a first flared portion coupled with a distal end of the stent body and spaced around a circumference of the distal end of the stent body, and wherein the second anchoring member comprises a second flared portion coupled with a proximal end of the stent body and spaced around a circumference of the proximal end of the stent body.

3. The stent of claim 2, wherein a length the first anchoring member is different than a length of the second anchoring member.

4. The stent of claim 2, wherein a length the first anchoring member is the same as a length of the second anchoring member.

5. The stent of claim 3, wherein the first material is disposed onto an entire portion of the stent body, the first anchoring member, and the second anchoring member.

6. The stent of claim 5, wherein the helical wrapping pattern comprises a single wire frame.

7. The stent of claim 6, wherein the first material comprises a plurality of drainage holes disposed within the second anchoring member and the proximal portion of the stent body.

8. The stent of claim 7, further comprising:
one or more markers disposed around the stent body, the first anchoring member, and the second anchoring member.

9. The stent of claim 5, wherein the helical wrapping pattern comprises more than one wire frame.

10. The stent of claim 2, wherein the helical wrapping pattern comprises a laser cut frame.

11. The stent of claim 1, wherein the stent body further comprises a braided wrapping pattern coupled with a distal end of the helical wrapping pattern, wherein the braided wrapping pattern comprises a braided frame.

12. The stent of claim 11, wherein the helical wrapping pattern comprises a single wire frame.

13. The stent of claim 11, wherein the first anchoring member comprises a flared portion coupled with the distal end of the stent body and spaced around a circumference of the distal end of the stent body, wherein the first anchoring member comprises the braided wrapping pattern.

14. The stent of claim 13, wherein the braided wrapping pattern is uncovered from the first material.

15. The stent of claim 1, wherein the helical wrapping pattern comprises a first wire frame around a first portion of a circumference of the stem body and a second wire frame around a second portion of the circumference of the stent body, wherein the first wire frame and the second wire frame are connected across the length of the stent body.

16. The stent of claim 15, wherein the first anchoring member comprises a first fin that protrudes from the distal portion of the stent body and extends in a proximal direction or a distal direction, and wherein the second anchoring member comprises a second fin that protrudes from the proximal portion of the stent body and extends in the proximal direction or the distal direction.

17. The stent of claim 15, wherein the first material is disposed onto an entire portion of the stent body, the first anchoring member, and the second anchoring member.
